(19) United States
(12) Patent Application Publication
(10) Pub. No.: US 2003/0192555 A1
(43) Pub. Date: Oct. 16, 2003

(54) Direct arterial infiltration for production of vascular pathology

(76) Inventors: Elazer Edelman, Brookline, MA (US); Campbell Rogers, Westwood, MA (US); Frederick G. Welt, Newton, MA (US)

Correspondence Address: Attn: Matthew E. Connors, Samuels, Gauthier & Stevens, LLP, Suite 3300, 225 Franklin Street, Boston, MA, 02110, US

(21) Appl. No.: 10/429,418

(22) Filed: May 5, 2003

Related U.S. Application Data
(62) Division of application No. 09/627,752, filed on Jul. 28, 2000, now abandoned.
(60) Provisional application No. 60/146,622, filed on Jul. 30, 1999.

(51) Int. Cl. A61B 19/00

(52) U.S. Cl. 128/898

(57) ABSTRACT

A method of producing a vascular lesion in an animal that resembles atherosclerosic lesions in humans. The method includes introducing cholesterol enriched with LDL or cholesterol enriched with LDL and monocytes, macrophages, leukocytes, smooth muscle cells or platelets into a proliferative lesion created by standard methods, to promote atherosclerosis.

### BACKGROUND OF THE INVENTION

The invention relates to the field of the local infiltration of cells, cell products or chemical and pharmacological compounds from the lumen of a tubular structure into or around the wall of that structure. In particular this invention is for the infusion of cells, cell products or chemical and pharmacological compounds, alone or in some form of a polymeric material, from an endovascular catheter into or around the wall of a blood vessel. The infusion can be utilized to produce an animal model of atherosclerosis, and in particular to create a vascular lesion in an animal model that more closely resembles the lesions of atherosclerosis seen in humans as well as to the possibility of introducing cellular constituents, cell products or pharmacological agents in order to ameliorate the atherosclerotic or restenotic process. The infusion can also be used to treat the blood vessel wall through the infusion of cells that elaborate therapeutic compounds.

Animal models have been invaluable in understanding the basic processes of vascular biology in atherosclerotic and restenotic lesions. However, countless examples of therapies that are advantageous in animal models prove unsuccessful in humans. This has led to skepticism as to the degree to which current experimental models predict human responses. The differences are usually attributed to the inter-species differences in biologic responses.

Atherogenesis is the formation of lipid deposits in the intima of arteries that can cause the arterial surface to bulge out into the arterial lumen. In humans, atherogenesis is the result of many different factors acting over a prolonged period of time. Initial lesions called fatty streaks may develop early in life and are manifest by the presence of lipids and macrophages in the innermost layer of the vessel wall. There are many atherogenic determinants including prolonged exposure to factors such as hyperlipidemia, diabetes, cigarette smoking, and hypertension, which lead to further inflammation, endothelial activation, smooth muscle cell proliferation, matrix synthesis, monocyte recruitment, and eventually necrosis, lipid accumulation, and often, calcification. The mature atherosclerotic plaque contains two main components; the first is a soft, lipid-rich core with necrotic debris. The second is a fibrous cap separating the core from the lumen and consisting of smooth muscle cells and collagen. It remains unclear whether this lipid core develops by direct infiltration and deposition of extracellular lipid with subsequent recruitment of macrophages, or whether macrophages endocytose lipid and subsequently necrose leaving lipid and necrotic debris behind. The oxidative state of the lipid core is crucial in determining it's toxic potential.

Vascular lesions created in an animal model do not closely resemble the lesions of atherosclerosis seen in humans. Thus, there is a need for an animal model of atherosclerosis that more faithfully reproduces human conditions in order to study and understand the atherosclerotic process and to design and test strategies to ameliorate the process. Current animal models of atherosclerosis are successful to varying and limited degrees.

Non-human primates have developed lesions that are similar in their histo-pathology to those of humans. However, the time and cost required for such primate models, has made them unsuitable for routine animal investigations. Rabbit or swine hypercholesterolemic models have also been employed, either using animals with endogenous hyperlipidemia or by feeding normal animals hyperlipidemic diets. Predominantly proliferative atheromatous lesions develop over time consisting of abundant smooth muscle cells interspersed with macrophages, but they lack the lipid core and fibrous cap seen in mature human atherosclerotic lesions. In an attempt to accelerate this process, several methods of endothelial injury have been employed including surface desiccation, electrical stimulation, and endothelial denudation (e.g. withdrawal of an inflated balloon within the artery). With these techniques, predominantly proliferative atheromatous lesions with modest monocyte accumulation can be produced in a matter of weeks. However, the complicated, mature atherosclerotic plaque has not been reproduced. While many of the central elements of the atherosclerotic process are present in animal models, there are crucial factors missing, including the soft lipid rich core and it's associated burden of lipid-rich macrophages.

Restenosis is the process of rapid reblockage of arteries following percutaneous coronary intervention. Human pathologic studies have revealed that this is predominantly a cellular proliferative lesion. In animal models, simple removal of the endothelium, an accompanying phenomenon of angioplasty, is a potent stimulus for proliferation on its own. Re-introduction of endothelial cells in the form of a perivascular implant can significantly reduce intimal growth suggesting that introduction of cellular constituents can help modulate vascular repair.

### SUMMARY OF THE INVENTION

A method for producing a more human-like vascular lesion. The method includes feeding an animal a hyperlipidemic diet, denuding arterial segments of the animal to produce a proliferative lesion, and introducing cholesterol enriched with LDL or cholesterol enriched with LDL and macrophrages into the proliferative lesion to promote atherosclerosis. A method of modulating the response to vascular injury necessarily created during percutaneous coronary intervention by injecting cellular constituents directly into the vascular wall.

An object of the present invention is to provide a vascular lesion, which resembles the lesions of atherosclerosis in humans.

A further object is to provide a method of producing the vascular lesion.

A further object is to provide a mechanism in which cells and/or cellular components (such as endothelial cells) may be delivered into or around the wall of the artery in order to help ameliorate the atherosclerotic or restenotic process directly or through the delivery of secreted substances.

Advantageously, the vascular lesions can be become an essential tool for exploring the biological mechanisms of atherosclerosis, and be an invaluable aid in testing interventions against these lesions.

These and other objects, features and advantages of the present invention will become apparent in light of the following detailed description of preferred embodiments thereof.

### DETAILED DESCRIPTION OF THE INVENTION

1. Atherosclerotic Model

With the shortcoming of current animal models, an atherosclerotic lesion may be created in a variety of animal models such that they more closely resemble mature human atheroma. The models may be used to answer fundamental questions regarding the roles of elements within the atheroma. The atherosclerotic lesions are created by introducing the components of the atherosclerotic lesion that are currently missing from animal models directly into the arterial wall, namely, the lipid core and associated macrophages. This lipid core takes years to accumulate in humans and is the defining component of the mature plaque. This method would obviate the need for prolonged administration of a cholesterol enriched diet by placing the lipid directly within the arterial wall and would impart characteristics of the mature atheromatous plaque as well.

Animals are to be fed a hyperlipidemic diet (2-3% peanut oil enriched with 0.5-2.0% cholesterol by weight) for 4 weeks. Under anesthesia, arterial segments will be denuded using Fogarty balloon catheters. This will produce a proliferative lesion consisting mainly of smooth muscle cells. The animals will be allowed to recover and at 14 days will again be anesthetized and either approximately 100-400 microliters of cholesterol enriched with LDL, or cholesterol enriched with LDL and macrophages, or other leukocytes, smooth muscle cells, or platelets are introduced into the proliferative lesion. A catheter such as the “Infiltrator” available from Inter Ventional Technologies Incorporated of Sand Diego, Calif. will deliver the substances directly into the arterial wall in a non-traumatic fashion. The Infiltrator catheter is designed to efficiently introduce microliter quantities of material into the arterial wall through the use of miniature injector ports under low pressure. Animals can be harvested at various time points in order to examine these lesions in a detailed histologic manner.

The materials are injected by themselves, together with other materials or encapsulated within or on hydrophilic or hydrophobic, non-erodible or bioerodible, homogeneous or heterogeneous polymeric materials, such as alginates, pluronics, hydrogels, EVAc, polystyrene, pLA, pGA or copolymers thereof. The polymeric materials may be in the form of a gel, foam, solid mass, homogeneous or heterogeneous matrix or solution, and they may be in their pure form or coated with a cell adhesion modifying material, chemical or biological sequence or element. Also, the materials may be injected into the wall or through and outside a portion or the entire wall.

The arterial segments may also be manipulated to augment injury with endovascular insertion and manipulation of wire loops, coils or filaments, insufflation with air, external compression or electrical stimulation, application of chemicals, temperature extremes or energy, or the placement of temporary or permanent implants such as endovascular stents. The animals can be selected from a group consisting of those animals with genetic predisposition to disease or those provided with dietary supplementation that induces disease.

2. Cellular Implants

While the invention above is described by the infusion of cholesterol and macrophages, it should be noted that a variety of substances or cell-types could be infused into or around the arterial wall in order to better understand the atherosclerotic process and to create more human-like atheroma. In addition, the process could also be used to deliver cells such as endothelial cells that have a natural or induced ability to limit cellular proliferation and therefore prevent excessive arterial narrowing.

Cells (endothelial and other types) are grown to confluency through standard cell-culture techniques. They are trypsinized in order to cause their detachment and placed in a solution of phosphate buffered or normal saline. The solutions may be approximately 10-15 million/ml but may differ depending on the material that was selected. They then can be delivered through a device such as the Infiltrator Catheter from Interventional Technologies Inc. Cells can also be embedded within polymeric foams, gels, matrices, or solutions or implanted on the surface of materials and then injected in and/or around a blood vessel.

The cells, their constituents or products may be selected from the group consisting of leukocytes, monocytes, macrophages, eosinophils, basophils, polymorphonucleur leukocytes, smooth muscle cells, endothelial cells, platelets, osteoclasts, osteoblasts, cartilage, or bone. Adjunctive compounds that might be injected along with, before or after the injection of cells, cell constituents or products might, include EDTA, lipopolysaccharide, oils, lipids, fats, or triglycerides. Further more, the cell constituents may be selected from the group consisting of DNA, oligonucleotides, mitochondria, and biochemical compounds such as proteins, polysaccharides, prostaglandins, or endothelial-derived constructing factor.

The lesions that are produced using the above method may be utilized to test methods of healing lesions within the walls of tubular tissues through the introduction of cells, cell elements and chemical or pharmacological compounds from the lumen of the tubular tissue into or around the wall of the tubular tissue. The tissue is an element of the cardiovascular, gastrointestinal, genitourinary, respiratory, or nervous systems, wherein the tubular tissue is subjected to intervention including mechanical, chemical, pharmacological, temperature or energy application. The infused cells include endothelial cells, leukocytes, monocytes, macrophages, smooth muscle cells, platelets, or genetically engineering cells. The genetically engineering cells secrete factors, compounds, cellular elements that inhibit smooth muscle cell proliferation, migration, or transformation, inflammation, vascular remodeling, thrombosis, or tissue hyperplasia.

Although the present invention has been shown and described with respect to several preferred embodiments thereof, various changes, omissions and additions to the form and detail thereof, may be made therein, without departing from the spirit and scope of the invention.

What is claimed is:

1. A method of producing a vascular lesion in an animal that resembles atherosclerosic lesions in humans, said method comprising:
introducing materials such as cholesterol enriched with LDL or cholesterol enriched with LDL and monocytes, macrophages, leukocytes, smooth muscle cells or platelets into or around a proliferative lesion to promote atherosclerosis.

2. The method of claim 1, wherein arterial segments are subject to balloon catheter inflation, surface denudation with for example wire loops, coils or filaments, insufflated air, external compression or electrical stimulation, chemical, temperature or energy injury, or the placement of temporary permanent implants such as endovascular stents.

3. The method of claim 1, wherein the animal is a native form of the animal or selected from the group consisting essentially of animals with genetic predisposition to disease or dietary supplementation that induces disease.

4. A method of producing a vascular lesion in an animal, which resembles atherosclerosic lesions in humans, said method comprising:
feeding the animal a hyperlipidemic diet;
denuding arterial segments of the animal to produce a proliferative lesion; and
introducing cells into said proliferative lesion to promote atherosclerosis.

5. The method of claim 4, wherein the cells may be selected from the group consisting of leukocytes, monocytes, macrophages, eosinophils, basophils, polymorphonucleur leukocytes, smooth muscle cells, endothelial cells, platelets, osteoclasts, osteoblasts, cartilage, or bone.

6. The method of claim 4, wherein the cell constituents may be selected from the group consisting of DNA, oligonucleotides, mitochondria, biochemical compounds such as proteins, polysaccharides, prostaglandins, or endothelial-derived constructing factor.

7. The method of claim 4, wherein chemical compounds may be infused with, before or after the cells, said compounds may be selected from the group consisting of chemical compounds such as EDTA, lipopolysaccharide, oils, lipids, fats, or triglycerides.

8. The method of claim 1, wherein the materials are injected by themselves, together with other materials or encapsulated within or on hydrophilic or hydrophobic, non-erodible or bioerodible, homogeneous or heterogeneous polymeric materials such as alginates, pluronics, hydrogels, EVAc, pLA, pGA or copolymers thereof.

9. The method of claim 8, wherein the material is injected into the wall or through and outside the wall.

10. The method of claim 8, wherein the polymeric material is in the form of a gel, foam, solid mass, homogeneous or heterogeneous matrix or solution, in its pure form, some combination of these materials or coated with a cell adhesion modifying material, chemical or biological sequence or element.

11. A method of healing lesions within the walls of tubular tissues through the introduction of cells, cell elements and chemical or pharmacological compounds from the lumen of the tubular tissue into the wall of the tubular tissue.

12. The method of claim 11, wherein said tissue is an element of the cardiovascular, gastrointestinal, genitourinary, respiratory, or nervous systems.

13. The method of claim 11, wherein said tubular tissue is subject to intervention including mechanical, chemical, pharmacological, temperature or energy application.

14. The method of claim 11, wherein infused cells include endothelial cells, leukocytes, monocytes, macrophages, smooth muscle cells, platelets, or genetically engineering cells.

15. The method of claim 14, wherein genetically engineering cells secrete factors, compounds, cellular elements that inhibit smooth muscle cell proliferation, migration, or transformation, inflammation, vascular remodeling, thrombosis, or tissue hyperplasia.
